(19) United States
(12) Patent Application Publication
(10) Pub. No.: US 2022/0193128 A1
(43) Pub. Date: Jun. 23, 2022

(54) A COMPOSITION, A KIT AND USE THEREOF

(71) Applicant: THERAVET SA, Charleroi (BE)

(72) Inventors: Enrico BASTIANELLI, Rhode-Saint-Genèse (BE); Julie WINAND, Marche les Dames (BE); Caroline ZEIPPEN, Flostoy (BE); Sabrina ENA, Montigny le Tilleul (BE)

(21) Appl. No.: 17/624,828

(22) PCT Filed: Jul. 7, 2020
(86) PCT No.: PCT/EP2020/069134

§ 371 Date: Jan. 4, 2022

(30) Foreign Application Priority Data
Jul. 8, 2019 (EP) 19184942.1

(51) Int. Cl. A61K 35/16; A61K 31/728; A61K 9/00; A61K 38/36; A61K 31/4168; A61K 33/06; A61K 35/28; A61K 35/32; A61K 47/02; A61P 19/02

(52) U.S. Cl. CPC A61K 35/16; A61K 31/728; A61K 9/0019; A61K 38/363; A61P 19/02; A61K 33/06; A61K 35/28; A61K 35/32; A61K 47/02; A61K 31/4168

(57) ABSTRACT

The current invention relates to a composition for parenteral administration comprising a mammalian plasma and hyaluronic acid or a salt or ester thereof, wherein said plasma comprises lipids and/or phospholipids. The inventions also relates to a kit comprising one or more aliquots of a composition, said composition comprising a mammalian plasma comprising lipids and/or phospholipids and hyaluronic acid or a derivative thereof and optionally one or more pharmaceutical active ingredients, wherein said kit further comprises one or more aliquots of a calcium source, preferably a calcium chloride solution. The invention also pertains to the composition and kit of current invention for use in the treatment of musculoskeletal diseases.

### FIELD OF THE INVENTION

The present invention relates to a composition for parenteral administration, a kit comprising the composition. The invention also relates to the composition and kit for use in the treatment of musculoskeletal diseases. The present invention pertains to the technical field of medical and veterinary sciences.

### BACKGROUND OF THE INVENTION

Musculoskeletal disorders are a large group of common disorders which can affect bones, muscles, ligaments, tendons, cartilage and joints. Musculoskeletal disorders occur frequently and include non-specific complaints. Pain is the most common symptom related to musculoskeletal disorders, which may be caused by the progressed inflammation. Often nonsteroidal anti-inflammatories are used to treat the inflammation or pain. Besides pain, other symptoms such as stiffness, tenderness, weakness and swelling or deformity of affected parts are manifestations of musculoskeletal disorders. In essence, musculoskeletal diseases are difficult to diagnose and/or treat.

Various therapies improve to a certain extent the overall quality of life of a subject suffering from the disease. The symptoms are managed by providing nonsteroidal anti-inflammatories, local analgesic therapies, or intra-articular corticosteroid injection. In some cases, surgery is applied. However, the outcome of the therapy is often adverse.

Plasma therapies whether in combination with regenerative medicine or not, is a recent approach for treating musculoskeletal disorders. These plasma therapies can be provided in a pharmaceutical acceptable formulation.

EP 2 900 247 discloses a pharmaceutical formulation comprising solvent/detergent-treated plasma and hyaluronic acid.

EP 2 185 163 discloses a method for the treatment of a joint, said method comprises the infiltration of a compound in the joint, wherein said compound comprises at least one blood-derived substance.

WO 2013 076 160 discloses a gel-forming formulation comprising a glycosaminoglycan, a sustained release agent and one or more pharmaceutical active ingredients.

WO2009/016451 describes methods for treating articular diseases or pain, based on joint infiltration with blood-derived substances such as PRP and plasma rich in growth factors.

DE10054257 describes a composition for intra-articular injection, comprising oxygen-activated plasma. An analog of proteoglycans is used (pentosan polysulfate). DE10054257 further mentions the presence of phospholipids such as phosphatidylcholine.

US2012171169 describes pharmaceutical compositions for the prevention of bone and cartilage diseases. The composition comprises adipose-derived stem cells, PRP, HA and calcium chloride.

WO2014049063 finally describes a composition of a solvent treated plasma (lipid-free) with HA and calcium.

The gel-forming compositions disclosed in above mentioned documents display or attain a gel consistency upon administration. There however remains a need for further and/or improved pharmaceutical formulations configured for local administration, which have an optimal viscoelastic properties, improved stability, better jellification, cell-supportive properties, and biocompatibility.

The aim of the invention is to provide an improved pharmaceutical formulation configured for local administration.

### SUMMARY OF THE INVENTION

The present invention and embodiments thereof serve to provide a solution to one or more of above-mentioned disadvantages. To this end, the present invention relates to a composition according to claim 1. Preferred embodiments of the composition are shown in any of the claims 2 to 13.

In a second aspect, the present invention relates to a kit according to claim 14. Preferred embodiments of the kit are shown in claim 15.

In a third aspect the present invention relates to the composition and kit of current invention for use according to claim 16. Preferred embodiments of this use are shown in claim 17-20.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” as used herein are synonymous with “include”, “including”, “includes” or “contain”, “containing” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “wt. %” here and throughout the description unless otherwise defined, refers to the relative weight of the respective component based on the overall weight of the formulation.

Whereas the terms “one or more”, such as one or more member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

The term “plasma” is to be understood as a fraction obtained from a sample of whole blood, provided or contacted with an anticoagulant, e.g., CPDA-1 (comprising citrate, phosphate, dextrose, and/or adenosine) heparin, citrate, oxalate or EDTA. Cellular components of the blood sample, i.e. white and red blood cells are separated from the liquid component, i.e. plasma, by an appropriate technique e.g., centrifugation or apheresis. Afterwards, the plasma may optionally be solvent/detergent treated, e.g. to eliminate the viruses. Solvent/detergent treatment enables the elimination of cells and/or cells debris e.g., lipid membranes, lipids and/or phospholipids. This is typically referred to as solvent/detergent treated plasma (S/D plasma). Plasma that did not undergo the solvent/detergent treatment can be labeled as non-treated plasma or native plasma. In an embodiment of the current invention, the plasma is non-treated plasma. Without wishing to be bound to a theory, said plasma is understood to comprise cells, lipids and/or phospholipids. As a consequence, said plasma has similar or the same compositional characteristics with the native plasma. The “plasma” of current invention is preferably mammalian derived.

The term “fresh frozen plasma” is to be understood as a plasma frozen within twelve hours at or below −18° C. following its preparation as referred in the plasma definition.

The term “apheresed plasma” is to be understood as a plasma obtained by plasmapheresis, a medical technology in which the blood is collected from the donor, passed through an apparatus (i.e. an apheresis) that separated the plasma from the all blood and returns the remainder fluid directly to the donor. Plasma that did undergo apheresis are contacted with anti-coagulant such as CPDA 1, ACD, heparin, citrate, oxalate or EDTA.

The term “centrifuged plasma” is to be understood as a plasma obtained by centrifugation of the all blood contacted with an anti-coagulant allowing the separation of the plasma from the all blood. Plasma that did undergo centrifugation can be contacted with anti-coagulant such as CPDA 1, ACD, heparin, citrate, oxalate or EDTA.

The term “hyaluronic acid” may be used interchangeably with “hyaluronate”. The term “hyaluronic acid” refers to an anionic, non-sulfated polymer of disaccharides composed of D-glucuronic acid and N-acetyl-D-glucosamine, linked via alternating β-1,4 and β-1,3 glycosidic bonds. Hyaluronic acid derivatives include but are not limited to salts of hyaluronate such as sodium hyaluronate or an ester of hyaluronic acid with an alcohol of the aliphatic, heterocyclic or cycloaliphatic series, or a sulphated form of hyaluronic acid or combination of agents containing hyaluronic acid.

The term “subject” or “patient” are used interchangeably and refer to animals, preferably vertebrates, more preferably warm-blooded animals, even more preferably mammals. Preferred patients are equine, bovine, porcine, ovine, canine, or feline subjects.

The term “a subject in need of treatment” includes subjects that would benefit from treatment of a given condition, particularly of a musculoskeletal disease such as a bone disease or a joint disease. Alternatively or in addition, said disease may affect tendons and/or ligaments. Such subjects may include, without limitation, those that have been diagnosed with said condition, those prone to develop said condition and/or those in whom said condition is to be prevented.

The term “treatment” encompasses both the therapeutic treatment of an already developed disease or condition, as well as prophylactic or preventative measures, wherein the aim is to prevent or lessen the chances of incidence of an undesired affliction, such as to prevent the chances of progression of the disease or condition. Beneficial or desired clinical results may include, without limitation, alleviation of one or more symptoms or one or more biological markers, diminishment of extent of disease, stabilized state of disease, delay or slowing of disease progression, amelioration or palliation of the disease state, and the like. The term “treatment” can also mean prolonging survival as compared with expected survival if not receiving treatment.

The terms “sustained release” as used herein, broadly refer to the release of a compound from a composition over an extended, prolonged or increased period of time compared with the release of said compound from a reference composition such as a composition known in the prior art. As used herein, the sustained release refers to the prolonged release of one or more of the components of the composition, namely of the mammalian plasma, or of one or more additional active pharmaceutical ingredient(s). For instance, it is known from the prior art that the half-life of high molecular weight hyaluronic acid in the knee joint is about 10 to 15 hours. The sustained release, as used herein, thus refers to the extended release of a hyaluronic acid from the present composition, for example release during one or more days, such as during 2 days, 3 days, 4 days, 5 days, 6 days, or during one or more weeks such as during 1.5 week, 2 weeks, 3 weeks, or during one or more months. These terms may thus also specifically encompass extended release, delayed release or controlled release.

The term “pharmaceutical compound” broadly refers to a compound, substance or component which, when provided in an effective amount, achieves a desired therapeutic and/or prophylactic outcome(s). Typically, an active pharmaceutical ingredient may achieve such outcome(s) through interacting with and/or modulating living cells or organisms. The term “pharmaceutical compound” also encompasses any pharmacologically active salts, esters, N-oxides or prodrugs of the title compound or substance.

The term “local administration” as used herein refers to a parenteral administration in or in the vicinity of a targeted site within the body.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### DETAILED DESCRIPTION OF THE INVENTION

The present invention relates to a composition for parenteral administration, comprising mammalian derived plasma and hyaluronic acid or a derivative thereof. The composition is safe and efficient, improving the physiological and pathological responses or effects in mammals. Furthermore, the composition is independent of the mammalian subject whereto the composition is administered, underlining the overall biocompatibility and the ease of availability of the composition. The invention especially relates to plasma lipids and/or phospholipids to said composition. The invention further relates to a kit and use of the composition and kit.

In a first aspect, the invention provides a composition for parenteral administration comprising a mammalian plasma and hyaluronic acid or a derivative thereof, wherein said plasma comprises lipids and/or phospholipids.

The composition of current invention has great jellification properties due to the presence of plasma lipids and/or phospholipids, which present a pro-coagulation activity in presence of calcium. In an embodiment, the composition can be allogeneic used and can thus independent of the mammalian subject from which the composition obtained be administered, underlining the overall biocompatibility of the composition. It was found that the composition is well-tolerated by the subject, as no or hardly any adverse effects were observed.

In an embodiment of the composition, said composition comprises about 50 to 99 wt. % plasma, more preferably about 60 to 99 wt. %, more preferably about 70 to 99 wt. %, more preferably about 80 to 99 wt. %, more preferably about 90 to 99 wt. %, more preferably about 90, 91, 92, 93, 94, 95, 96, 97, 98, 99 wt. % plasma.

In an embodiment, a main component of the composition is mammalian derived plasma.

In a preferred embodiment of the invention, said plasma is mammalian derived, more preferably derived from equine, bovine, porcine, ovine, canine, or feline blood.

Preferably, the plasma may be mammalian plasma, such that the composition comprising mammalian plasma are particularly suited for administration to mammalian subjects. Preferably the plasma is allogeneic. The collection process of allogeneic plasma is well-established and highly standardized with regard to the use of anticoagulant, separation and processing techniques, centrifugal force, and temperature and time, resulting in highly predictable amount of cells and solid components. Preferably the plasma is allogeneic equine, bovine, porcine, ovine, canine, or feline plasma.

In an embodiment, said mammalian plasma preferably includes centrifuged plasma or apheresed plasma. The mammalian plasma may be fresh frozen plasma. Preferably the mammalian plasma of current invention is apheresed fresh frozen plasma.

In an embodiment, said plasma is a non-solvent/detergent treated plasma.

Mammalian derived plasma includes plasma collected from single, or multiple donors from mammalian sources, such as equine, bovine, porcine, ovine, canine, or feline in plastic bags, or glass bottles and separated from blood cells via the means described above. After collection, mammalian derived plasma is stored at or below −18° C., preferably below −18° C.

Plasma comprises lipids and/or phospholipids, which may include, but are not limited to one or more sphingolipids, phosphatidylserines, phosphatidylcholines, phosphatidylethanolamines, cholesterol, cholesterol esters, triglycerides, diacylglycerides, lecithin, liposomes, minor plasma lipids and combinations thereof.

While the lipids and/or phospholipids may be externally added to the composition, in an embodiment said plasma will be the source of the lipids.

Various analytical methods are available for the determination of plasma lipid and/or phospholipid concentrations and characteristics. Chromatography is an analytical procedure for separating and analyzing lipids. Thin layer chromatography (TLC), gas chromatography (GC), and high-pressure liquid chromatography (HPLC) are often used for the assessment of plasma lipids and/or phospholipids. Also spectrometry is a known technique to assess the plasma lipids and/or phospholipids in plasma, like mass spectrometry or nuclear magnetic resonance (NMR) spectroscopy. Enzyme-linked immunosorbent assay (ELISA) is also a technique to assess plasma lipids and/or phospholipids in plasma.

The plasma lipids and/or phospholipids are preferably quantified by using HPLC technique. Limits are set on the amount of lipids and/or phospholipids within the plasma of current invention enabling quality reproducibility of the plasma.

In an embodiment of the composition, the plasma comprises lipids and/or phospholipids, wherein said total concentration of lipids and/or phospholipids is between 0.2 and 7 mg/ml.

Preferably the total concentration of lipids and/or phospholipids in the plasma is between 0.4 and 6 mg/ml, more preferably between 0.5 and 5 mg/ml.

Said total concentration of lipids and/or phospholipids in the plasma enables an optimal jellification in the presence of a calcium source. Lower concentrations result in an insufficient jellification, whereas higher concentrations are no longer safe for use in prophylactic or therapeutic treatment.

In a further embodiment, said plasma comprises minor plasma lipids.

In a preferred embodiment of current invention, said minor plasma lipids are chosen from the group of palmitoyl ethanolamide (PEA), stearoyl ethanolamide (SEA), arachidonoyl ethanolamide (AEA).

It was found that the presence of this level of (phospho)lipids in the plasma provides coagulation activities. In an embodiment, the total concentration of minor plasma lipids in the mammalian plasma is between 5 and 50 nmol/l, more preferably between 6 and 30 nmol/l.

In a more preferred embodiment of the invention, said composition comprises PEA, SEA and AEA, wherein each lipid is present at a concentration of between 0.2 and 14 nmol/l. In a further embodiment, each minor plasma lipid is present at a concentration between 0.5 and 12 nmol/l, more preferably between 0.8 and 8.5 nmol/l. Lipids and/or phospholipids promote complex formation with blood coagulation factors triggering the blood coagulation cascade. The apheresed plasma of current invention comprises minor plasma lipids. Without wishing to be bound to a theory, said lipids are understood to have pro-coagulant activities in plasma and allow at least partially an improved jellification of the gel-forming composition.

Once the composition for parenteral administration is contacted with calcium, the composition may provide a natural environment for the surrounding cells and/or tissues and displays a particularly good gel-forming capacity. For example, once mixed with a calcium source and injected in articular cartilage defects, the present composition allows jellification and restores the physiological and rheological states of an affected area, like an arthritic joint. Further, these viscoelastic properties can ensure localized delivery and/or sustained release of components of the composition, namely of the plasma, which can comprise beneficial biological substances and/or of the hyaluronic acid.

The jellification activity of plasma can be measured by assessing the coagulation parameters, preferably PTT and aPTT, and/or coagulation factors, preferable factor VIII and fibrinogen.

In an embodiment, the plasma used has an anti-hemophilic factor (factor VIII) activity higher than 75%. Assays to quantify factor VIII activity are known in the art and may include clot-based assays, chromogenic assays or immunoassays such as ELISA. In an embodiment, said anti-hemophilic factor (factor VIII) activity is measured by a clot detection method using a viscosity-based detection system such as the STart® semi-automated benchtop analyzer or the automated STA R Max® 2, both from Stago (Cupaiolo R, Govaerts D, Blauwaert M, Cauchie P. Performance evaluation of a new Stago® automated haemostasis analyser: The STA R Max® 2. Int J Lab Hematol. 2019; 41(6):731-737. doi:10.1111/ijlh.13100). In short, said method uses the increase in viscosity of the plasma being tested for clot detection. The change in viscosity is measured by monitoring the amplitude of an oscillating steel ball in a specially designed cuvette. Movement of the steel ball is mediated by two activating coils, working alternatively to induce and maintain a natural oscillation. When the start reagent is added, the detection and the chronometer start immediately and simultaneously. As the ball oscillates left and right, the amplitude of the motion is measured. The chronometer times the clotting of the sample. Amplitude is monitored during the entire clotting process. The amplitude remains constant while no clot is present. As the clot develops, the viscosity increases and the amplitude decreases. An algorithm is used to determine the clotting time.

In an embodiment, the plasma used has a fibrinogen (factor I) concentration ranging between 0.25 and 3 g/l. In an embodiment, the plasma used has an activated partial thromboplastin time (aPTT) activity ranging between 12 and 65 sec and/or a partial thromboplastin time (PTT) activity ranging between 5 and 17 sec. Suited method to measure (a)PTT is by means of a clot detection method using a viscosity-based detection system such as the STart® semi-automated benchtop analyzer of Stago, which is described above. In a further preferred embodiment, said plasma complies with all of the above features. Assays to quantify factor VIII, fibrinogen concentration, aPTT activity, and PTT activity are known by a person skilled in the art.

Fibrinogen circulates as a soluble plasma glycoprotein in the plasma. Fibrinogen is involved in the formation of fibrin and promotes blood clotting by forming bridges and activating blood platelets through binding.

Preferably the mammalian plasma of current invention has a total concentration of fibrinogen of between 0.25 and 3 g/l, more preferably of between 0.3 and 2.5 g/l, more preferably of between 0.4 and 2.1 g/l. Prior art documents prefer a defibrinated plasma, current invention however requires mammalian plasma with a minimal concentration of fibrinogen of 0.25 g/l.

The total protein concentration of the mammalian plasma of current invention is preferably between 10 and 100 g/l, preferably between 15 and 85 g/l, more preferably between 20 and 80 g/l. It is believed that protein levels above this concentration may lead to problems with clogging when administered to a subject.

In a more preferred embodiment said plasma has a concentration of white blood cells (WBCs) of less than 200 mil/l, preferably a concentration of WBCs of between 3 and 145 mil/l, more preferably between 5 and 85 mil/l, desirably between 10 and 50 mil/l.

The mammalian derived plasma comprises white blood cells, also called leukocytes, in an amount that does not interfere with the jellification of the composition.

In a particularly preferred embodiment said plasma has a concentration of blood platelets (BPs) of less than 50,000 mil/l, preferably a concentration of BPs of between 1,000 and 35,000 mil/l, more preferably a concentration of BPs of between 2,000 and 20,000 mil/l.

Blood platelets, also called thrombocytes, help in the blood coagulation process. Furthermore, platelets contain hundreds of proteins, known as growth factors. These platelet-derived growth factors are useful in regenerative medicine, in particular for stimulating repair of bone, cartilage, tendon or ligaments defects or for replacing damaged bones, cartilages, tendons or ligaments. The platelet concentration is lower than in whole blood. Although other documents in the prior art prefer the use of platelet-rich plasma to enhance wound healing, the concentration of platelets is specifically lower. Said concentration of platelets improves the jellification process.

A second component of the composition is one or more glycosaminoglycans. In certain embodiments, the glycosaminoglycan may be selected from the group consisting of hyaluronic acid and derivatives thereof, a proteoglycan and derivatives thereof, a chondroitin sulfate, a keratan sulfate, a chitosan and derivatives thereof, a chitin and derivatives thereof; or mixtures of the aforementioned.

In a preferred embodiment said glycosaminoglycan is hyaluronic acid or a derivative thereof. Hyaluronic acid plays an important role in the biological organism, firstly as a mechanical support of the cells of many tissues, such as the skin, the tendons, the muscles and cartilage and it is therefore the main component of the extracellular matrix. But hyaluronic acid also performs other functions in the biological processes, such as the hydration of tissues, lubrication, cellular migration, cell function and differentiation.

In the current composition, the presence of hyaluronic acid will aid in biological processes such as tissue hydration, proteoglycan organization, cell differentiation, cell proliferation and angiogenesis. Hyaluronic acid derivatives maintain all the properties of said glycosaminoglycan, with the advantage of being able to be processed in various forms and having solubility and degradation times which, vary according to the type and percentage of derivation. Derivatives of hyaluronic acid may be a salt of hyaluronic acid, an ester of hyaluronic acid with an alcohol of the aliphatic, heterocyclic or cycloaliphatic series, or a sulphated form of hyaluronic acid. Without limitation, suitable derivatives may be salts of hyaluronic acid, such as preferably sodium hyaluronate.

The hyaluronic acid or derivative thereof preferably has a molecular weight of less than 1,800 kDa. Particularly preferred may be hyaluronic acid or derivatives thereof with a molecular weight of between 700 and 1,600 kDa, more preferably between 700 and 1,000 kDa.

Preferably the hyaluronic acid or a derivative thereof has a high molecular weight. The high molecular weight of hyaluronic acid or derivative should be understood as a molecular weight of between 700 and 1,000 kDa. For example, injecting hyaluronic acid of such a high molecular weight is effective for restoring the mechanical integrity of joints affected by osteoarthritis. Particularly preferred, the hyaluronic acid or derivative thereof has a narrow size distribution. It was observed that the molecular weight ranges as disclosed above ensures a suitable affinity for hyaluronic acid receptors and a better stimulation of the hyaluronic acid biosynthesis. Desirably, the hyaluronic acid or derivative thereof has a high molecular weight and a narrow size distribution. Furthermore, hyaluronic acid or derivative thereof with a molecular weight of between 700 and 1,000 kDa improves the cell viability of the surrounding cells once implanted or injected in the tissue in need of a treatment.

The composition of current invention has great viscoelastic properties and presents satisfactory scaffold stability due to the narrow size distribution and high molecular weight of the hyaluronic acid. These properties are of interest, especially in view of the necessity of hyaluronic acid or a derivative thereof to absorb major variations in pressure which occur in vivo in joints.

Preferably the hyaluronic acid or derivative thereof is an unbranched straight-chain hyaluronic acid.

Hyaluronic acid occurs naturally and does not have a species or organ specificity, even when implanted or injected into a living body. Hyaluronic acid shows excellent biocompatibility. Hence, the hyaluronic acid could be animal derived or of biofermentative origin. A well-known source of hyaluronic acid in the art is a rooster comb. Preferably the hyaluronic acid is of biofermentative origin.

Without limitation, the composition may comprise the hyaluronic acid or derivative thereof in a concentration ranging from about 0.10 to 200 mg/ml, preferably from about 1 to 100 mg/ml, more preferably from about 2 to 50 mg/ml, more preferably from about 4 to 20 mg/ml.

In an embodiment, the hyaluronic acid or derivative thereof is preferably sterilized. Hyaluronic acid can be steam sterilized or filter sterilized. Preferably the hyaluronic acid is filter sterilized, as steam sterilization may affect the molecular weight distribution range of the hyaluronic acid fibers. The hyaluronic acid is preferably solubilized and sterilized by filtration.

Preferably the mammalian plasma is filtered through a sterile filter.

The filter sterilization of the mammalian plasma and hyaluronic acid has beneficial influences on the stability and activity of the plasma and hyaluronic acid.

The composition of current invention, comprising the mammalian plasma and hyaluronic acid or derivative thereof are preferably freeze-dried. The mammalian derived plasma and hyaluronic acid or derivative thereof are admixed, resulting in a plasma and hyaluronic acid mixture. Once the mixture is made and freeze-dried, a lyophilized product is obtained. The following terms “lyophilized product” and “freeze-dried mixture” are used interchangeably herein.

In a preferred embodiment, the plasma and hyaluronic mixture is diluted prior to freeze-drying. Preferably said mixture is solubilized at least 4 times, preferably at least 3 times, more preferably at least 2 times, desirably 2 times. Said solubilization improves the quality of the plasma and hyaluronic acid mixture in the composition.

The freeze-drying of said mixture increases the shelf-life. Freeze-drying of said mixture provides a lyophilized and stable product for months. The lyophilized product can be easily stored, shipped and reconstituted for injection. The lyophilized product is stable at a temperature of between 15 and 25° C. Having a lyophilized product with a stability at room temperature is beneficial for the medical practitioner. The product can be easily transported or handled, without substantially deteriorating the quality, safety and activity of the product. There is no need for special a cooling container.

In certain embodiments, the composition may comprise one or more pharmaceutical compounds in addition to the mammalian plasma and hyaluronic acid or derivative thereof. In other embodiments, the plasma and hyaluronic acid or derivative thereof may be the only components of the composition; hence, in such embodiments the composition may consist of or consist essentially of the plasma and hyaluronic acid or derivative thereof.

Depending on the nature of the pharmaceutical compound, said compound may be added at different timing within the preparation of the composition. In an embodiment, said pharmaceutical compounds are mixed with the plasma and hyaluronic acid mixture. In an embodiment, said pharmaceutical compounds are mixed with the plasma and hyaluronic acid mixture prior to freeze-drying said mixture. In an embodiment, said pharmaceutical compounds are mixed with the freeze-dried plasma and hyaluronic acid mixture. In a preferred embodiment, said pharmaceutical compounds are mixed with the plasma and hyaluronic acid mixture, freeze-dried or not, prior to the addition of the calcium source.

In certain embodiments, the composition may advantageously further comprise one or more pharmaceutical compounds. The applicability of the present invention is not limited to any pharmaceutical compound or class of pharmaceutical compound. The pharmaceutical compound may be pharmacologically active itself, or may be converted into a pharmacologically active species by a chemical or enzymatic process in the body, i.e., the pharmaceutical compound may be a prodrug. The current gel-forming composition may be particularly useful for poorly-stable pharmaceutical compounds. Illustrative non-limiting examples of poorly-stable pharmaceutical compounds include peptides and proteins such as growth factors, peptide-like active ingredients, antibodies and vaccines, small interfering RNA (siRNA), DNA, hormones, etc.

Moreover, combination of two or more pharmaceutical compounds or dose combinations may be included as the drug component. In this case, the release of each compound may be identical or different such as for instance in case of a combination of two compounds in which the first one is presented as an immediate release form and the second one as a controlled release. Similarly, a combination of immediate release and controlled release form may also be obtained for the same compound, in order to provide a rapid and sustained effect.

In an embodiment of current invention said gel-forming composition further comprises one or more pharmaceutical compounds, wherein said compounds are selected from the group consisting of an active pharmaceutical ingredient, an antibiotic agent, a cell composition, a small organic molecule, a protein, or a peptide.

In an embodiment, said active pharmaceutical ingredient is an alpha-2 adrenergic receptor agonist, preferably clonidine or a derivative thereof, preferably clonidine.

In a specific embodiment the alpha-2 adrenergic receptor agonist may be selected from the group consisting of clonidine and derivatives thereof, including 2,6-dimethylclonidine, 4-azidoclonidine, 4-carboxyclonidine-methyl 3,5-dichlorotyrosine, 4-hydroxyclonidine, 4-iodoclonidine, alinidine, apraclonidine, chlorethylclonidine, clonidine 4-isothiocyanate, clonidine 4-methylisothiocyanate, clonidine receptor, clonidine-displacing substance, hydroxyphenacetyl aminoclonidine, N,N′-dimethylclonidine, p-aminoclonidine, and tiamenidine; imidazolidines, including imidazolines, impromidine, detomidine, medetomidine, dexmedetomidine, levamisole, losartane, lofexidine, miconazole, naphazoline, niridazole, nitroimidazoles, ondansetron, oxymetazoline, phentolamine, tetramisole, thiamazole, tizanidine, tolazoline, trimetaphan; imidazoles, including 4-(3-butoxy-4-methoxybenzyl) imidazolidin-2-one, urocanic acid, amino-imidazole carboxamide, antazoline, biotine, bis (4-methyl-1-homo piperazinylthiocarbonyl) disulfide, carbimazole, cimetidine, clotrimazole, creatinine, dacarbazine, dexmedetomidine, econazole, enoximone, ethymizol, etomidate, fadrozole, fluspirilene, idazoxan, mivazerol; guanidines, including agmatine, betanidine, biguanides, cimetidine, creatine, gabexate, guanethidine, guanethidine sulfate, guanclofine, guanfacine, guanidine, guanoxabenz, impromidine, iodo-3 benzylguanidine, methylguanidine, mitoguazone, nitrosoguanidines, pinacidil, robenidine, sulfaguanidine, zanamivir; alpha-methyinorepherine, azepexole, 5-bromo-6-(2 imidazolidine-2-ylamino) quinoxalin, formoterol fumarate, indoramin, 6-allyl-2-amino-5,6,7,8-tetrahydro4H-thiazolo [4,5-d]azepine diHCl, nicergoline, rilmenidine, N-(2-bromo-6-fluorophenyl)-4,5-dihydro-1H-imidazol-2-amine, and xylazine.

Clonidine is a centrally acting alpha-2 adrenergic receptor agonist. It is generally used as an anti-hypertensive agent. Clonidine is also commonly referred to as 2,6-dichloro-N-2-imidazolidinyldenebenzenamine (C₉H₉Cl₂N₃). The composition of current invention further comprising clonidine allows for a sustained drug release. The composition of current invention comprises clonidine in a therapeutically effective amount. The concentration of clonidine in the composition is preferably between 0.01 and 0.5 mg/ml, more preferably between 0.03 and 0.3 mg/ml, more preferably between 0.05 and 0.2 mg/ml. Said low concentrations of clonidine or a derivative thereof is effective in the administered tissue. The composition further improves the patient disease-specific quality of life.

In a preferred embodiment said cell composition comprises one or more antibiotic agents. In the context of the present invention, an antibiotic agent is a substance that has the capacity to inhibit the growth of or to destroy bacteria and other microorganisms. In a more preferred embodiment the antibiotic agent is selected from the classes consisting of beta-lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, antibiotic steroids, sulfonamides, tetracycline, dicarboxylic acids, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds. Preferably the antibiotic agent is an aminoglycoside, preferably gentamicine.

In another embodiment, said composition may be further supplemented with a cell composition, said cell composition may be chosen from mesenchymal stem cells, osteoprogenitor cells, osteoblasts, osteocytes, chondroblasts and/or chondrocytes.

The mesenchymal stem cells (MSC) are capable of undergoing osteogenic or chondrogenic differentiation. Other cells are already committed towards osteoblastic or chondroblastic lineage. These cell compositions improve the treatment of musculoskeletal diseases, in particular bone or joint diseases. Preferably, the cells of the cell composition of current invention may be animal cells, preferably warm-blooded animal cells, more preferably mammalian cells. The mammalian cells are preferably of equine, bovine, porcine, ovine, canine, or feline origin.

Cell compositions become an alternative therapy of several physiological deficiencies. Without limitation, the composition may comprise an amount of cells ranging from about 0.05×10⁶ to 5×10⁹ cells, preferably from about 0.5×10⁶ to 1×10⁹ cells, more preferably from about 4×10⁶ to 250×10⁶ cells.

The composition of current invention allows for delivery of such a cell composition. The composition of current invention is an improved composition protecting the cells from the physical, surrounding environment once injected. The composition ensures a suitable supportive environment for the cells. The viscoelastic properties of the composition stabilize the cells and allow integration and propagation of the cells at the specific site of injury. Furthermore, the composition of current invention stabilizes the cells such that the formation of cell aggregates in the composition are obstructed, allowing the administration of a sufficient amount of single cells within the site of injury, thereby improving the tissue regeneration, specifically the musculoskeletal tissue regeneration.

In an embodiment, said cell composition is added after the lyophilization of the plasma and glycosaminoglycan mixture in order to prevent disruption of said cells.

In another and further embodiment of current invention, said small organic molecule is a scaffold or matrix component with osteoconductive properties, preferably tricalcium phosphate particles (TCP).

The osteoconductive matrices further allow for a sustained release of the pharmaceutical compounds, like for instance clonidine, in the composition of current invention and promote bone repair. The matrix allows influx of the cells as discussed above facilitating bone formation and repair of the fracture site. Tricalcium phosphate particles are mineral materials effective to provide a scaffold or matrix for bone ingrowth. Preferably TCP have an average particle diameter between about 5 and 200 μm, more typically between about 10 and 100 μm, and desirably between about 20 and 60 μm. Methods to measure the particle size of TCP are known in the art and may include sedimentometry, analytical centrifugation and various kinds of spectrometry. In an embodiment, said TCP particle size is measured by laser diffraction. The concentration of TCP in the composition is preferably between 10 and 200 mg/ml, more preferably between 30 and 150 mg/ml, more preferably between 50 and 100 mg/ml. The osteoconductive matrices of current invention can take on any shape and are malleable, cohesive and can be injected at or near the target tissue site.

The composition of current invention may additionally comprise proteins or (poly)peptides. Biologically active proteins, peptides and polypeptides suitable for administration in the composition of current invention include growth hormones, growth factors, and other biologically active fragments and derivatives thereof. Preferred proteins include equine, bovine, porcine, ovine, canine, or feline growth hormones; and is meant to encompass those which are of natural, synthetic, recombinant or biosynthetic origin. Additionally, metals or metal compounds associated with biologically active proteins, peptides and polypeptides, as well as acid salts, derivatives and complexes and anti-hydrating agents are suitable for incorporation into the composition of the invention.

The present composition may comprise, in addition to the herein particularly specified pharmaceutical compounds, one or more pharmaceutical excipients. Suitable excipients depend on the dosage form and identities of said compounds and can be selected by the skilled person. Pharmaceutical excipients should not interfere with the activity of the plasma, hyaluronic acid or pharmaceutical compounds.

In an embodiment, the composition may further comprise whole blood or a fractionated component of whole blood. The addition of whole blood or said fractionated component, preferably of whole blood, to the present composition may allow at least partially to improve the regenerative properties. The composition comprising whole blood or said fractionated component thereof advantageously comprise platelet-derived growth factors useful in regenerative medicine, in particular for stimulating repair of bone, cartilage, tendon or ligaments defects or for replacing damaged bones, cartilages, tendons or ligaments. For example, the whole blood may be allogeneic or autologous with respect to the subject receiving the formulation.

In an embodiment, the composition is devoid or substantially devoid of serum.

In another embodiment, the composition may further comprise one or more substance with osteogenic, osteo-inductive and/or osteo-conductive properties. In preferred embodiments, such substance may be selected from the group comprising or consisting of a fibroblast growth factor (FGF), preferably FGF-2, a transforming growth factor beta (TGFB), preferably TGFB-1, platelet-derived growth factor (PDGF), interleukin-8 (IL-8), a bone morphogenetic protein (BMP), for example any one or more of BMP-2, BMP-4, BMP-6 and BMP-7, parathyroid hormone (PTH), parathyroid hormone-related protein (PTHrp), and stem cell factor (SCF). Hence, in certain embodiments, the pharmaceutical active protein or peptide may be a growth factor, preferably a growth factor selected from the group consisting of a FGF, a TGFB, PDGF, IL-8, a BMP, PTH, PTHrp, and SCF, more preferably a growth factor selected from the group consisting of FGF-2, TGFB-1, PDGF, IL-8, BMP-2, BMP-4, BMP-6, BMP-7, PTH, PTHrp, and SCF.

As mentioned earlier in current invention, the composition comprising the mammalian plasma and hyaluronic acid or derivative thereof and optionally one or more pharmaceutical compounds such as an alpha-2 adrenergic receptor are in a form, which can easily be reconstituted by adding an optimum volume of an aqueous solution. Said plasma and hyaluronic acid mixture supplemented with clonidine are preferably solubilized twice in aqueous solution and lyophilized in one master batch. Preferably said master batch is dispatched in small containers. It was found that the solubilization and subsequent lyophilization resulted in an aerated product which is easily and fast resuspended in an aqueous solution. Resuspension can go as fast as within 15 minutes, even 10 minutes.

By preference, the composition of current invention is suitable for parenteral administration and will finally thus be administered in aqueous form.

In another embodiment, said freeze-dried mixture may be supplemented with a jellification aid such as a calcium source.

Said calcium source may be selected from a suitable amount of pharmaceutically acceptable calcium salt(s), preferably soluble calcium salt(s). Such Ca²⁺ salts may be formed with inorganic or organic acids. Examples of such salts include calcium chloride (CaCl₂)), calcium glycerophosphate, calcium phosphate, calcium hydrogen carbonate, calcium citrate, calcium sulphate, calcium lactate, calcium gluconate, calcium ascorbate, and mixtures thereof. Particularly preferred is calcium chloride, which displays advantageously good solubility and is well-tolerated in injectable solutions.

In an embodiment said calcium source is a calcium solution. Preferably, the calcium solution is prepared by adding a source of calcium, preferable calcium chloride to an acidic solution, such as for instance a hydrogen chloride solution. Preferably the final pH of the acidic solution in the composition is between 1 and 4, more preferably between 1.3 and 2.1, more preferably between 1.5 and 2.5.

Said acidic solutions are preferably pharmaceutically acceptable acids. Pharmaceutically acceptable acids may include, but are not limited to (i) an inorganic acid such as hydrochloric acid, hydrobromic acid, hydroiodic acid and the like, (ii) an organic mono-, di- or tri-carboxylic acid (for example, formic acid, acetic acid, adipic acid, alginic acid, citric acid, ascorbic acid, aspartic acid, benzoic acid, butyric acid, camphoric acid, gluconic acid, glucuronic acid, galacturonic acid, glutamic acid, heptanoic acid, hexanoic acid, fumaric acid, lactic acid, lactobionic acid, malonic acid, maleic acid, nicotinic acid, oxalic acid, pamoic acid, pectinic acid, 3-phenylpropionic acid, picric acid, pivalic acid, propionic acid, succinic acid, tartaric acid, undecanoic acid and the like) or (iii) a sulfonic acid (for example, benzenesulfonic acid, sodium bisulfate, sulfuric acid, camphorsulfonic acid, dodecylsulfonic acid, ethanesulfonic acid, methanesulfonic acid, isethionic acid, naphthalenesulfonic acid, p-toluenesulfonic acid and the like).

In a preferred embodiment of the invention said calcium source is a calcium chloride solution. In a particularly preferred embodiment of the invention said calcium chloride solution has an acidic pH preferably between a pH of 1 and 4. Said calcium chloride solution is preferably admixed with the freeze-dried mixture to dissolve the mixture prior to administration. Said calcium chloride solution enables the formation of a gel with high cohesive properties, allowing an optimal integration of the gel-forming composition at the site of injury. Once admixed, the pH of the composition is physiologic, preferably between a pH of 5 and 8, more preferably between a pH of 6 and 7.8.

In a second aspect current invention pertains to a kit, wherein the kit comprises one or more aliquots of a composition, said composition comprising a mammalian derived plasma and a hyaluronic acid or a derivative thereof and optionally one or more pharmaceutical compounds, selected from the group consisting of an active pharmaceutical ingredient, a cell composition, a small organic molecule, a protein, or a peptide, wherein said plasma comprises lipids and/or phospholipids wherein said composition is in a freeze-dried form, and wherein said kit further comprises one or more aliquots of a calcium source, preferably a calcium chloride solution.

The kit according to present invention simplifies the preparation of the composition. This simplification results in a reduced chance of contamination and erroneous concentration. In addition, the kit makes it possible for the medical or veterinary practitioner to prepare a composition wherein a controlled jellification takes place.

The kit comprises aliquots or other containers suitable for biological liquids like the plasma, the hyaluronic acid, the calcium, the cells. The components to obtain the composition for parenteral administration of current invention are provided in a package convenient for distribution to a practitioner of skill in the art. The kit further comprises means for administering the composition to the subject in need of the treatment.

Preferably, said plasma comprises lipids and/or phospholipids. More preferably, said plasma comprises minor plasma lipids. Said minor plasma lipids are preferably chosen from the group of palmitoyl ethanolamide (PEA), stearoyl ethanolamide (SEA), arachidonoyl ethanolamide (AEA).

The kit provides one or more aliquots of calcium chloride such that the total concentration of calcium chloride in the composition is between 0.2 and 1.4 mg/l. Preferably, the final calcium concentration in the composition after adding the calcium source is between 0.4 and 1.2 mg/l, more preferably between 0.6 and 1.0 mg/ml, more preferably 0.65 and 0.95 mg/ml, more preferably 0.7 and 0.9 mg/ml, more preferably 0.75 and 0.85 mg/ml.

In a preferred embodiment a sufficient amount of the composition is dissolved in a sufficient amount of said calcium source, thereby obtaining a solution, said solution is administered to a subject, said administration is preferably parenteral.

The solution is in the form of a parenterally acceptable aqueous solution, which is pyrogen-free and has suitable pH, isotonicity and stability.

The composition of current invention is dispersed in a pharmaceutically and pharmacologically acceptable liquid to obtain a slow release composition for parenteral administration, such as parenteral injection. Preferably the composition is configured for intraosseous, periosseous, intraarticular, periarticular administration, or for intratendon, peritendon, intraligament, or periligament administration.

These modes of administration will strongly depend on the site of injury, being for example a joint, ligament, tendon or tendon sheet. The site of delivery of the composition is typically at or near the site of tissue damage. The site of tissue damage is determined by well-established methods, including imaging studies, known by a skilled person.

In a particular embodiment the composition is configured for intraosseous or periosseous administration. Intra-osseous administration or delivery generally refers to a method whereby a treatment is delivered, directly or indirectly, into the bone (trabecular or cortical). Peri-osseous administration or delivery generally refers to a method whereby a treatment is delivered in the surroundings of a bone (especially around the fracture/damage site). In another particular embodiment, the composition is configured for intraarticular or periarticular administration. Intra-articular administration or delivery generally refers to a method whereby a treatment is delivered, directly or indirectly, into the synovial capsule of an articulating joint. Peri-articular administration or delivery generally refers to a method whereby a treatment is delivered in the surroundings of the synovial capsule of an articulating joint and/or the subchondral bone. In a further embodiment, the composition is configured for intratendon or peritendon administration. In another and further embodiment the composition is configured for intraligament or periligament administration. A person skilled in the art is familiar with the indications for and complications associated with these injection techniques.

The composition provides dosage forms of biologically active substances which release the substance in a controlled manner and, which reduces the frequency of administration. Without limitation, a typical dose of for instance the plasma and hyaluronic acid to be administered may range from about 0.1 to 2 ml per injection. The kit of current invention can be provided with prophylactic effective amounts and/or therapeutic effective amounts.

A qualified physician will be able to determine the dose and amount of injections taking into account the size of the patient. Dosage and administration are adjusted to provide sufficient levels of the active moiety or to maintain the desired effect.

A third aspect of current invention relates to a kit for use in the treatment of a musculoskeletal disease, preferably a bone disease or a joint disease.

The treatment of musculoskeletal disease in current invention includes therapeutic and/or preventative measures. A disorder of the musculoskeletal system may include a disorder of the joint, cartilage, ligament, tendon, tendon sheath or a combination of different connective tissues.

Pathologies in the musculoskeletal system in present invention are, but not limited to (osteo)arthritis, tendinitis, fibromyalgia, bone fractures. These disorders or injuries can often be multifactorial; aging, trauma, mechanical forces, conformation, hormonal and genetic factors contributing to varying degrees.

Preferably, the composition and kit of current invention will be used in a subject in need of treatment of said musculoskeletal disease.

In addition, the use of the kit or the solution of current invention may alleviate the symptoms of a subject diagnosed with musculoskeletal disease.

In an embodiment, the current invention relates to a composition or kit for use in the treatment of osteoarthritis. Osteoarthritis is a progressively worsening inflammation of the joint caused by the deterioration of cartilage. In a healthy joint, cartilage acts as a cushion to allow the joint to move smoothly through its full range of motion. In cases of osteoarthritis, this cartilage cushion begins to break down because of factors such as age, injury, repetitive stress, disease or genetic predisposition. The loss of this protective cushion results in pain, inflammation, decreased range of motion, and the development of bone spurs. Diagnosis is typically based on signs and symptoms, with medical imaging and other tests used to support or rule out other problems.

In an embodiment, the current invention relates to a composition or kit for use in the prevention of cranial cruciate ligament (CCL) rupture. The cranial cruciate ligament is one of the most important stabilizers inside the knee joint, the middle joint in the back leg. Rupture of the cranial cruciate ligament is one of the most common reasons for hind limb lameness, pain, and subsequent knee arthritis. While the clinical signs associated with CCL rupture vary, the condition invariably causes rear limb dysfunction and pain. Trauma accounts for a minority of CCL ruptures, whereas progressive degeneration of the ligament has been attributed to a variety of factors that may be broadly classified as genetic, conformational, environmental, immune-mediated, and inflammatory. Diagnosing complete tears of the CCL is easily accomplished using a combination of gait observations, physical examination findings, and radiography (X-rays). By contrast, partial CCL tears may be more challenging to diagnose. Use of the current composition or kit for treating and/or preventing partial rupture of the cranial cruciate ligament in a subject such as an animal, significantly reduces signs of ligament degeneration and helps prevent complete cranial cruciate ligament rupture and reduces the incidence of contralateral disease in subjects with unilateral CCL rupture.

In an embodiment, the current invention relates to a composition or kit for use in the treatment of tendinopathies, such as supraspinatus tendinopathy and Achilles tendon rupture. Tendinopathy is a type of tendon disorder that results in pain, swelling, and impaired function. Supraspinatus tendinopathy is a term used to describe tears, calcifying tendinopathy, tendinosis and/or injuries in and around the tendon of the supraspinatus muscle, and is a cause of forelimb lameness. The supraspinatus is an important passive stabiliser of the shoulder joint, and is responsible for shoulder extension and advancing the limb. Injury to the tendon of the supraspinatus muscle causes inflammation. Tearing of the tendon fibers and the resulting inflammation can lead to mineralization and calcification of the tendon, which are a source of pain and lameness. The Achilles tendon's main function is rear-limb forward progression, and it contributes to passive support of the hock. The etiology of Achilles tendon injuries is usually traumatic. Depending on the trauma, the severity of the lesion may vary considerably, leading to stretching, small or partial lacerations or a complete rupture.

The treatments of the invention may comprise administration of a single therapeutically effective dose or administration of multiple therapeutically effective doses of the solution for parenteral administration.

In an embodiment, the composition according to the current invention is intraarticular or locally administered in a single or multiple therapeutically effective dose(s). The dose is adapted to the targeted joint and the body weight of the subject.

Specifically in the case of tendinopathies, the composition according to the current invention is periarticular, peritendinous or intratendinous administered in a single or multiple therapeutically effective dose(s). The dose is adapted to the targeted site and the body weight of the subject.

Also intended is the use of the composition as described above for the manufacture of a medicament for the treatment of a musculoskeletal disease, preferably a bone disease or a joint disease, more preferably osteoarthritis, prevention of CLL rupture or tendinopathies such as supraspinatus tendinopathy and Achilles tendon rupture.

The invention is further described by the following non-limiting examples which further illustrate the invention, and are not intended to, nor should they be interpreted to, limit the scope of the invention.

### EXAMPLES

The present invention will now be further exemplified with reference to the following example(s). The present invention is in no way limited to the given examples.

### Example 1: Compositional Characteristics of the Non-Solvent/Detergent Treated Canine (C) Plasma and Solvent/Detergent-Treated Canine Plasma (S/D) Plasma

TABLE 1
compositional characteristics of a plasma of current
invention and a S/D treated plasma of canine origin

| | | S/D plasma |
|---|---|---|
| Parameter | C plasma (n = 6) | (n = 5) |
| White blood cell concentration (mil/l) | 28.5 ± 13.2 | 0 |
| Blood platelet concentration (mil/l) | 7866.7 ± 4195.7 | 0 |
| PTT activity (sec) | 8.7 ± 1.2 | 11 ± 0 |
| aPTT activity (sec) | 16.9 ± 0.8 | 30 ± 1 |
| Factor VIII activity (% activity) | 204 | 100 |
| Fibrinogen concentration (g/l) | 0.9 ± 0.3 | 2.6 ± 0.1 |
| Total protein concentration (g/l) | 34.2 ± 4.5 | 57 ± 4 |
| Total lipid concentration (g/l) | 4 ± 2 | 0 |

Reviewing the composition of C and S/D plasma, S/D plasma shows a significant absence of cells, lipids in comparison to C plasma due to the solvent/detergent treatment. The lipids present a specific coagulation activity, which improves the jellification of the composition. The activity of PTT, aPTT and factor VIII is measured using a clot-based detection system, more specifically the STart® semi-automated benchtop analyzer of Stago.

### Example 2: Comparative Example of S/D Treated Plasma and Non-S/D Treated Plasma

A comparative experiment was performed wherein the jellification of a composition comprising a plasma with lipids (non S/D treated plasma) and a composition comprising a plasma without lipids (S/D treated plasma) was compared. Both compositions comprise 10 mg/ml sodium hyaluronate and 0.10 mg/ml clonidine. Table 2 gives an overview of the different tested calcium concentrations on the composition with S/D treated plasma or non S/D treated plasma.

TABLE 2
jellfication of a composition of current invention in comparison to a composition
with S/D treated plasma with increasing calcium concentrations.

| | Calcium concentration | | | | |
|---|---|---|---|---|---|
| | 0.5 mg/ml | 0.8 mg/ml | 0.9 mg/ml | 1 mg/ml | 2 mg/ml |
| Composition | No | No | No | No | No |
| with S/D | jellification | jellification | jellification | jellification | jellification |
| treated | | | | | |
| plasma | | | | | |
| (n = 10) | | | | | |
| Composition | No | 20 ± 6 | 16 ± 3 | 15 ± 3 | 8 ± 5 |
| with non | jellification | minutes | minutes | minutes | minutes |
| S/D treated | | | | | |
| plasma | | | | | |
| (n = 10) | | | | | |

As presented in Table 2, the calcium alone triggers the jellification of the composition with non S/D treated plasma. Said jellification may also occur in the presence of physiological fluid and/or cells.

The composition with S/D treated plasma needs other additional ingredients to initiate the jellification of the composition, which could interfere with the biological substances within the composition. Furthermore, these additional ingredients could induce an immune response, as these may be foreign to the immune system.

The lower calcium concentrations result in a controlled jellification of the composition and give the medical practitioner a suitable timeframe to prepare the composition for parenteral administration maintaining the activity, safety and efficacy of the composition.

### Example 3: Case Study of Great Danes with Osteoarthritis

Dogs often suffer from arthritis because of excessive running or exercise, injury and/or genetic predisposition. In this case study five male Great Danes suffering from osteoarthritis in the hip joint were treated with the gel-forming composition of current invention. All dogs were over 4 years of age and presented stiffness in the hip joint, as well as alterations in the hip joint structures. In addition, a decreased flexibility was assessed and severe pain was ensued due to the lack of hydration and inflammation of the joint. Furthermore, cartilage defects were noticed on the CT and radiography. A kit according to current invention was used for the treatment of the hip joint. A dose of solubilized calcium chloride provided in the kit of current invention was admixed with a dose of canine plasma and hyaluronic acid mix. A total volume of 1 ml was locally administered at injured hip joint. After injection all dogs were followed by daily examination of the injected joint. The dogs were closely monitored. Particular attention was paid to observing any possible adverse effects or hypersensitivity reaction, local warmth, sweating, heavy breathing or fever. No adverse effects were noted, except for one Great Dane which showed local swelling the first 24 hours after the administration of the product. Further evaluation of the dogs during the rehabilitation showed an improved functionality and sustainability of the damaged joint.

### Example 4: Different Final Product Compositions

Compositions comprising a mammalian derived plasma and hyaluronic acid or a derivative thereof and optionally one or more pharmaceutical active ingredients according to any of the embodiments as described above are suitable for parenteral administration. Without limitation, compositions of current invention are listed.

### Composition I:

99.5% canine plasma
9 mg/ml hyaluronic acid
60 mg/ml TCP
0.15 mg/ml clonidine
0.7 mg/ml calcium chloride
26.04 mg/ml citric acid

### Composition II:

99% equine plasma
2 mg/ml hyaluronic acid
0.6 mg/ml calcium chloride
0.55 mg/ml HCl

### Composition III:

99% canine plasma
10 mg/ml sodium hyaluronate
0.10 mg/ml clonidine
0.8 mg/ml CaCl₂)
0.55 mg/ml HCl

### Composition IV:

98% feline plasma
15 mg/ml hyaluronic acid
1.2 mg/ml calcium chloride
0.55 mg/ml HCl

### Composition V:

99% canine plasma
10 mg/ml sodium hyaluronate
0.8 mg/ml CaCl₂)
0.88 mg/ml ascorbic acid

### Composition VI:

99% canine plasma
10 mg/ml sodium hyaluronate
0.10 mg/ml clonidine HCl
diluted in autologous canine whole blood

### Example 5: Efficacy Evaluation of a Single Intra-Articular Administration of a Composition According to the Current Invention in an Induced Osteoarthritis Model in Young Adult Beagle Dogs: A 3-Month Follow-Up Study

In this study sixteen young adult Beagle dogs were subjected to a complete transection of the cranial cruciate ligament of the right paw to induce osteoarthritis in the stifle joint. Fifteen days following surgery study animals were randomly allocated to experiment groups according the following table:

| Experimental | | Number and |
|---|---|---|
| Groups | Treatment | gender of Animals |
| Control | Hyaluronic acid (HA) solution (10 ml/ml) | 8 males |
| Composition A | 10 mg/ml HA, 100 μg/ml clonidine HCl in | 8 males |
| | solution in canine plasma, 0.8 mg/ml | |
| | CaCl₂, 15 mM HCl | |

Treatment was administered 2 weeks after surgery by intra-articular administration of Control solution or Composition A.

In this example Composition A is a freeze-dried composition according to the current invention, which is resuspended in 2.5 ml of resuspension solution, comprising 10 mg/ml of hyaluronic acid, 100 μg/ml of clonidine HCl in solution in canine plasma, 0.8 mg/ml CaCl₂) and 15 mM HCl.

Treatment volume administered in each animal was calculated based on the body weight of the study animal and was comprised between 1 and 1.5 ml.

The efficacy endpoints of this study were (i) the evaluation of lameness using a gait walkaway system and (ii) the evaluation of radiological signs of osteoarthritis using X-Ray images.

The measurement and observations were done before surgery, before treatment (2 weeks after surgery) and then 1-, 2- and 3-months following intra-articular administration of Control or Composition A. This study showed that pressure parameters related to lameness such as % TPI, Total Scaled Pressure and GLS (Gait4Dog® lameness score) were statistically significantly improved overtime in the group receiving Composition A as compared to Control. Pressure parameters were increased of about 50% at 3-month follow-up in the group receiving the Composition A as compared to Control.

Additionally, osteoarthritis score evaluated based on X-Ray imaging was statistically significantly reduced by 15% and 13% at 2- and 3-month follow-up in the group receiving Composition A as compared to Control.

The data from this study show the effect of Composition A on clinical signs of OA from 2 months following IA administration: (i) by improving lameness as compared to Control treatment and (ii) by slowing down the progression of radiological signs of OA. Therefore, the data of this study support the efficacy of the proposed therapeutic effective dose and treatment regimen for Composition A for the treatment of osteoarthritis.

1. A composition for parenteral administration comprising a mammalian plasma and hyaluronic acid or a salt or ester thereof, characterized in that said plasma comprises lipids and/or phospholipids.

2. The composition according to claim 1, characterized in that said lipids and/or phospholipids are present in the plasma at a total concentration of between 0.2 and 7 mg/ml.

3. The composition according to claim 1, characterized in that said plasma comprises minor plasma lipids, preferably at a total concentration of between 5 and 50 nmol/l.

4. The composition according to claim 2, characterized in that said minor plasma lipids are chosen from the group of palmitoyl ethanolamide (PEA), stearoyl ethanolamide (SEA), arachidonoyl ethanolamide (AEA).

5. The composition according to claim 1, characterized in that said plasma has an anti-hemophilic factor (factor VIII) activity higher than 75%

6. The composition according to claim 1, characterized in that said plasma has a fibrinogen concentration ranging between 0.25 and 3 g/l.

7. The composition according to claim 1, characterized in that said plasma has an activated partial thromboplastin time (aPTT) activity ranging between 12 and 65 sec and/or a partial thromboplastin time (PTT) activity ranging between 5 and 17 sec.

8. The composition according to claim 1, characterized in that said plasma is mammalian derived, more preferably derived from equine, bovine, porcine, ovine, canine, or feline blood.

9. The composition according to claim 1, characterized in that said composition further comprises one or more pharmaceutical compounds, wherein said compounds are selected from the group consisting of an active pharmaceutical ingredient, an antibiotic agent, a cell composition, a small organic molecule, a protein, and a peptide.

10. The composition according to claim 9, characterized in that said pharmaceutical active compound is an alpha-2 adrenergic receptor agonist, preferably clonidine or a derivative thereof, preferably clonidine.

11. The composition according to claim 1, characterized in that said composition further comprises a calcium source, preferably calcium chloride.

12. The composition according to claim 9, characterized in that said cell composition is chosen from mesenchymal stem cells, osteoprogenitor cells, osteoblasts, osteocytes, chondroblasts and/or chondrocytes.

13. The composition according to claim 9, characterized in that said small organic molecule is a scaffold or matrix component with osteoconductive properties, preferably tricalcium phosphate particles (TCP).

14. A kit comprising one or more aliquots of a composition, said composition comprising a mammalian plasma comprising lipids and/or phospholipids, and hyaluronic acid or a salt or ester thereof and optionally one or more pharmaceutical active ingredients, selected from the group consisting of a pharmaceutical active compound, a cell composition, or a small organic molecule, and wherein said composition is in a powder form, and wherein said kit further comprises one or more aliquots of a calcium source, preferably a calcium chloride solution.

15. The kit according to claim 14, characterized in that said plasma comprises minor plasma lipids, wherein said minor plasma lipids are chosen from the group of palmitoyl ethanolamide (PEA), stearoyl ethanolamide (SEA), arachidonoyl ethanolamide (AEA).

16. The kit according to claim 14 for use in the treatment of a musculoskeletal disease, preferably a bone disease or a joint disease.

17. The kit according to claim 16 for use in the treatment of osteoarthritis.

18. The kit according to claim 16 for use in the treatment or prevention of cranial cruciate ligament rupture.

19. The kit according to claim 16 for use in the treatment of tendinopathies.

20. The kit according to claim 16, wherein a sufficient amount of the composition is dissolved in a sufficient amount of a calcium source, thereby obtaining a solution, said solution is administered to a subject, said administration is preferably parenteral.
